(19) United States
(12) Patent Application Publication
(10) Pub. No.: US 2021/0228183 A1
(43) Pub. Date: Jul. 29, 2021

(54) DIAGNOSTIC ULTRASOUND APPARATUS

(71) Applicant: KONICA MINOLTA, INC., Tokyo (JP)

(72) Inventors: Tomohiko SATSUKA, Tokyo (JP); Masashi KUNITA, Yokohama-shi (JP)

(73) Assignee: KONICA MINOLTA, INC., Tokyo (JP)

(21) Appl. No.: 17/136,447

(22) Filed: Dec. 29, 2020

(30) Foreign Application Priority Data
Jan. 23, 2020 (JP) 2020-008864

(51) Int. Cl. A61B 8/00

(52) U.S. Cl. CPC A61B 8/4433; A61B 8/4444; A61B 8/56; A61B 8/4427

(57) ABSTRACT

A diagnostic ultrasound apparatus includes a body and an input/output port. The body generates an ultrasound image based on a received signal from an ultrasound probe that transmits and receives an ultrasound wave. The input/output port is connected to the body. The body includes a body-side connection part having a plurality of terminal connection ports. The input/output port includes a port-side connection part that collectively holds terminals corresponding to the terminal connection ports. The body-side connection part and the port-side connection part are connected by a connection method chosen between an indirect connection method via a connector and a direct connection method not via the connector.

### CROSS-REFERENCE TO RELATED APPLICATIONS

The entire disclosure of Japanese Patent Application No. 2020-008864 filed on Jan. 23, 2020 is incorporated herein by reference in its entirety.

### BACKGROUND

### Technological Field

The present disclosure relates to a diagnostic ultrasound apparatus.

### Description of the Related Art

An ultrasound can be performed repeatedly because it produces an ultrasound image showing the state of heartbeat or fetal movement by a simple operation of applying an ultrasound probe to the surface of a body and has accordingly high safety.

As an apparatus for ultrasounds, there has been known a diagnostic ultrasound apparatus having a portable body (main part) and a cart to which the body is attached. For example, in JP 2006-518252 A, there is proposed a configuration in which the body of a diagnostic ultrasound apparatus is connected to a mini-dock (cart) by stacking connectors so that the diagnostic ultrasound apparatus is used.

### SUMMARY

However, in JP 2006-518252 A, no matter whether the body is used alone or used together with the cart, the position of an input/output port with respect to the body is the same, and hence usability is low. Further, in order to use the diagnostic ultrasound apparatus, connectors are attached to the body one by one. This attaching work of the connectors takes much time, and hence workability is low.

Objects of the present disclosure include providing a diagnostic ultrasound apparatus in which the attaching work is easy and that has high usability.

In order to achieve at least one of the abovementioned objects, according to an aspect of the present disclosure, there is provided a diagnostic ultrasound apparatus including:

a body that generates an ultrasound image based on a received signal from an ultrasound probe that transmits and receives an ultrasound wave; and

an input/output port that is connected to the body,

wherein the body includes a body-side connection part having a plurality of terminal connection ports,

wherein the input/output port includes a port-side connection part that collectively holds terminals corresponding to the terminal connection ports, and

wherein the body-side connection part and the port-side connection part are connected by a connection method chosen between an indirect connection method via a connector and a direct connection method not via the connector.

### BRIEF DESCRIPTION OF THE DRAWINGS

The advantages and features provided by one or more embodiments of the present invention will become more fully understood from the detailed description given hereinbelow and the appended drawings that are given by way of illustration only, and thus are not intended as a definition of the limits of the invention, wherein:

FIG. 1 is a perspective view showing a body and a cart of a diagnostic ultrasound apparatus;

FIG. 2 is a block diagram showing a functional configuration of the cart;

FIG. 3 is a diagram for explaining the state of the diagnostic ultrasound apparatus at the time of use; and

FIG. 4 is a diagram for explaining the state of the diagnostic ultrasound apparatus at the time of use.

### DETAILED DESCRIPTION OF EMBODIMENTS

Hereinafter, one or more embodiments of the present invention will be described with reference to the drawings. However, the scope of the present invention is not limited to the disclosed embodiments or illustrated examples.

### [Configuration]

Configuration of a diagnostic ultrasound apparatus 1 according to an embodiment(s) will be described.

The diagnostic ultrasound apparatus 1 is installed in an examination room or the like of a medical institution, such as a hospital, and generates ultrasound images (tomograms, various signal waveforms, etc.) from echo signals corresponding to echoes from a subject(s) (patient(s)) by transmitting and receiving ultrasound waves to and from an ultrasound probe (not shown), which transmits and receives ultrasound waves, put on the subject. The diagnostic ultrasound apparatus 1 is configured to be movable so that a user, such as a doctor or a technician, can use it and also move with it in a medical institution, such as a hospital.

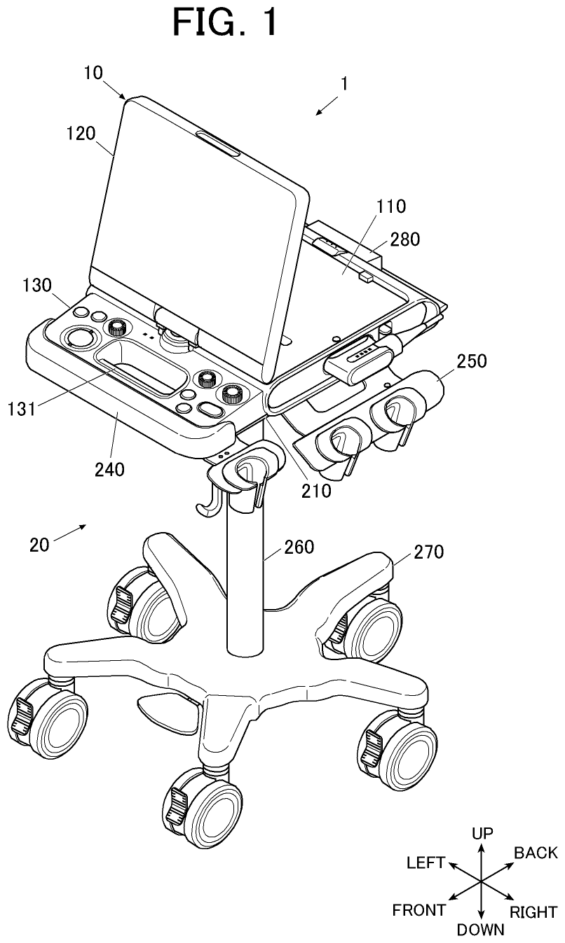

FIG. 1 is a perspective view showing the overall configuration of the diagnostic ultrasound apparatus 1.

As shown in FIG. 1, the diagnostic ultrasound apparatus 1 includes a body (main part) 10 and a cart 20 that is used by the user as needed. FIG. 1 shows the front-back direction, the right-left direction and the up-down direction in this application.

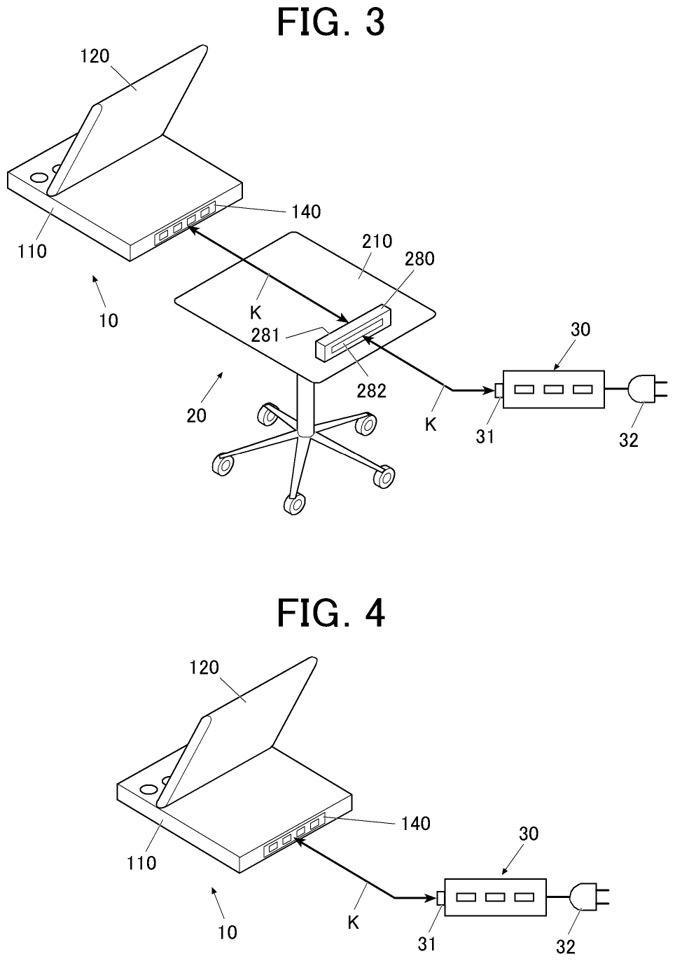

The body 10 transmits drive signals to the ultrasound probe, receives echo signals from the ultrasound probe as electric signals, and generates and displays ultrasound image data based on the echo signals. The cart 20 is a table on/to which the body 10 is mountable/attachable and that is freely movable by wheels. The body 10 is detachable from the cart 20 and portable. As described below, an input/output port 30 (shown in FIG. 3 and FIG. 4) is directly or indirectly connected to the body 10 so that the diagnostic ultrasound apparatus 1 is used.

The body 10 includes a case 110, a display 120, an operation unit 130 and a body-side connection part 140 (shown in FIG. 3 and FIG. 4).

The case 110 is shaped, for example, to be a substantially rectangular parallelepiped with at least the back end rounded, and has various electronic components, substrates and so forth built therein.

The display 120 is, for example, a foldable display that is connected to the case 110 and constituted by an LCD (Liquid Crystal Display), an ELD (Electro Luminescence Display) or the like, and displays display information, such as ultrasound images. Folding the display 120 makes it easier to carry the body 10.

The operation unit 130 includes various switches, buttons, a trackball, a mouse and a keyboard that are used for inputting a command(s) to start diagnosis (ultrasound(s)) and data, such as personal information of the subject, for example. The operation unit 130 has a handle 131 at the central portion in the right-left direction at the front for the user to grab.

The body-side connection part 140 is disposed on the lateral surface of the body 10 at the back. The body-side connection part 140 has a plurality of terminal connection ports. To the body-side connection part 140, a port-side connection part 31 of the input/output port 30 that collectively holds terminals corresponding to the terminal connection ports is directly or indirectly connectable.

The cart 20 includes a body mounting surface 210, a front-end guide part 240, an ultrasound probe holder 250, a support 260, a caster unit 270 and a connection unit 280.

The body mounting surface 210 is a plane perpendicular to the up-down direction, and a plane of the body 10 perpendicular to the up-down direction is mounted thereon. On the body mounting surface 210, a fixing unit (not shown) that fixes the body 10 to the body mounting surface 210 in the up-down direction and the right-left direction is disposed.

The front-end guide part 240 corresponds to the front of the body 10, and is disposed at the front end of the body mounting surface 210. The front-end guide part 240 can position the front end of the body 10.

The ultrasound probe holder 250 holds various ultrasound probes (not shown) connected to the body 10.

The support 260 is connected to the body mounting surface 210 and the caster unit 270, extends in the up-down direction, and supports the body 10 (body mounting surface 210, etc.).

The caster unit 270 has wheels, and can move the cart 20 and the mounted/attached body 10 in the front-back direction and the right-left direction by rotation of the wheels.

The connection unit 280 is disposed, on the body mounting surface 210, at a location corresponding to the back of the body 10.

The connection unit 280 is a unit into which a first connection part 281 that is connected to the body-side connection part 140 and a second connection part 282 that is connected to the port-side connection part 31 of the input/output port 30 are integrated.

In the state in which the body 10 is connected to the connection unit 280, the connection unit 280 can also function as a back-end guide part that positions the back end of the body 10.

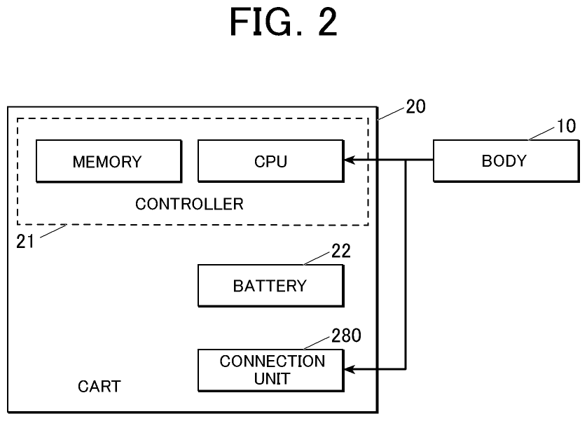

FIG. 2 is a block diagram showing a functional configuration of the cart 20.

As shown in FIG. 2, the cart 20 includes a controller 21 (hardware processor) including a CPU (Central Processing Unit) and a memory, and a battery 22.

The CPU is a processor that performs various types of arithmetic processing and controls operation of each component of the cart 20 overall. The CPU performs various types of control by reading and executing programs stored in the memory.

The memory provides the CPU with a memory space for working and stores various data. The memory includes, for example, a RAM (Random Access Memory) and a nonvolatile memory. The RAM is used for the arithmetic processing that is performed by the CPU, and also stores temporary data. The nonvolatile memory is, for example, a flash memory, and stores various settings, programs and so forth.

The battery 22 supplies power of a predetermined operation voltage to each component of the cart 20. The battery 22 includes, for example, a solar panel and a storage battery. As the battery 22, a detachable dry cell or button cell or the like may be used, or the battery 22 may have a secondary cell, such as a lithium-ion battery, which is charged by being connected to a power source with a predetermined cable or via a connection terminal.

As shown in FIG. 2, the control configuration of the cart 20 has a path giving access to the CPU and a path not giving access to the CPU when the body 10 and the input/output port 30 are connected.

The input/output port 30 includes the port-side connection part 31 that collectively holds terminals corresponding to the terminal connection ports. The port-side connection part 31 is connected to the body-side connection part 140 of the body 10.

The input/output port 30 has at least one of an external-device connection terminal, an image output terminal and a probe port for, for example, USB, DVI and HDMI products, such as cables, and the diagnostic ultrasound apparatus 1 can be used by any of these being connected to the input/output port 30.

The input/output port 30 includes a power source 32.

FIG. 3 and FIG. 4 are diagrams for explaining the state of the diagnostic ultrasound apparatus 1 at the time of use.

The diagnostic ultrasound apparatus 1 is used in the state in which the input/output port 30 is connected to the body 10.

As shown in FIG. 3, when the cart 20 is used, the body-side connection part 140 of the body 10 and the first connection part 281 of the connection unit 280 of the cart 20 are connected.

In addition, the port-side connection part 31 of the input/output port 30 and the second connection part 282 of the connection unit 280 of the cart 20 are connected.

At the time, (i) the body-side connection part 140 and the first connection part 281 and (ii) the port-side connection part 31 and the second connection part 282 each can be connected by direct insertion or via a predetermined connector K. As the connector(s) K, a connector with a cable can be used.

As shown in FIG. 4, when the cart 20 is not used, the body-side connection part 140 of the body 10 and the port-side connection part 31 of the input/output port 30 are connected.

At the time, the body-side connection part 140 and the port-side connection part 31 can be connected by direct insertion or via a predetermined connector K. As the connector K, a connector with a cable can be used.

The connection method of the body 10 and the input/output port 30 is determined by the user as the situation or the like at the time of use thereof demands. Thus, the user can appropriately adjust the position of the input/output port 30, and hence the usability is high.

Further, because the body-side connection part 140 has a plurality of terminal connection ports, and the port-side connection part 31 collectively holds terminals corresponding to the terminal connection ports, a plurality of terminals is collectively connectable, and hence the workability is high.

### Advantageous Effects

As described above, the diagnostic ultrasound apparatus 1 of this embodiment includes: the body 10 that generates an ultrasound image based on a received signal from an ultrasound probe that transmits and receives an ultrasound wave; and the input/output port 30 that is connected to the body 10.

The body 10 includes the body-side connection part 140 having a plurality of terminal connection ports, the input/output port 30 includes the port-side connection part 31 that collectively holds terminals corresponding to the terminal connection ports, and the body-side connection part 140 and the port-side connection part 31 are connected by a connection method chosen between the indirect connection method via the connector K and the direct connection method not via the connector K.

Hence, the attaching work is easy and the usability is high.

Further, according to this embodiment, the diagnostic ultrasound apparatus 1 further includes the cart 20 to which the body 10 is detachably attached, and the cart 20 includes: the first connection part 281 that is connected to the body-side connection part 140; and the second connection part 282 that is connected to the input/output port 30 (port-side connection part 31).

Hence, even when the cart 20 is used, the connecting work is easy, and also the usability is high.

Further, according to this embodiment, the first connection part 281 and the second connection part 282 are configured as the connection unit 280, into which the first connection part 281 and the second connection part 282 are integrated.

Hence, the configuration is simple.

### [Others]

The detailed configuration of each component constituting the diagnostic ultrasound apparatus 1 can be appropriately changed within a range not departing from the scope of the present disclosure.

For example, although in the above embodiment, the body-side connection part 140 is disposed at one location on the lateral surface of the body 10 at the back, the location(s) and the number of the body-side connection part(s) 140 are not limited thereto. For example, the body-side connection parts 140 may be disposed at two locations that are a location on the lateral surface of the body 10 at the back and a location on the lateral surface of the body 10 at the left. This enhances the degree of freedom at the time of use of the diagnostic ultrasound apparatus 1.

Further, although in the above embodiment, the connection unit 280 is disposed at one location on the body mounting surface 210 of the cart 20, the location(s) and the number of the connection unit(s) 280 are not limited thereto. For example, the connection units 280 may be disposed at two locations on the body mounting surface 210, the two locations corresponding to a side (right or left) and the back of the body 10. This further enhances the degree of freedom at the time of use of the diagnostic ultrasound apparatus 1.

Further, although in the above embodiment, the cart 20 includes the connection unit 280 into which the first connection part 281 and the second connection part 282 are integrated and that holds these together, the first connection part 281 and the second connection part 282 do not need to be formed as a unit and may be disposed at predetermined locations (e.g. at the front end and the back end of the cart 20).

Although one or more embodiments of the present invention have been described and illustrated in detail, the disclosed embodiments are made for purposes of not limitation but illustration and example only. The scope of the present invention should be interpreted by terms of the appended claims.

What is claimed is:

1. A diagnostic ultrasound apparatus comprising:
a body that generates an ultrasound image based on a received signal from an ultrasound probe that transmits and receives an ultrasound wave; and
an input/output port that is connected to the body,
wherein the body includes a body-side connection part having a plurality of terminal connection ports,
wherein the input/output port includes a port-side connection part that collectively holds terminals corresponding to the terminal connection ports, and
wherein the body-side connection part and the port-side connection part are connected by a connection method chosen between an indirect connection method via a connector and a direct connection method not via the connector.

2. The diagnostic ultrasound apparatus according to claim 1, wherein the body-side connection part of the body includes a plurality of body-side connection parts at a plurality of locations.

3. The diagnostic ultrasound apparatus according to claim 1, wherein the connector is a connector with a cable.

4. The diagnostic ultrasound apparatus according to claim 1, further comprising a cart to which the body is detachably attached, the cart including:
a first connection part that is connected to the body-side connection part; and
a second connection part that is connected to the port-side connection part.

5. The diagnostic ultrasound apparatus according to claim 4, wherein the first connection part and the second connection part are configured as a unit, into which the first connection part and the second connection part are integrated.

6. The diagnostic ultrasound apparatus according to claim 5, wherein the unit of the cart includes a plurality of units at a plurality of locations.

7. The diagnostic ultrasound apparatus according to claim 4, wherein the cart includes:
a hardware processor including a CPU that performs arithmetic processing; and
a path giving access to the CPU and a path not giving access to the CPU when the body-side connection part and the input/output port are connected.

8. The diagnostic ultrasound apparatus according to claim 1, wherein the input/output port includes a power source.

9. The diagnostic ultrasound apparatus according to claim 1, wherein the input/output port includes at least one of an external-device connection terminal, an image output terminal and a probe port.
